(19) United States
(12) Patent Application Publication
(10) Pub. No.: US 2007/0009956 A1
(43) Pub. Date: Jan. 11, 2007

(54) Arginine hydrochloride enhances chaperone-like activity of alpha crystallin

(75) Inventors: Volety Srinivas, Hyderabad (IN); Bakthisaran Raman, Hyderabad (IN); Kunchala Rao, Hyderabad (IN); Tangirala Ramakrishna, Hyderabad (IN); Chintalagiri Rao, Hyderabad (IN)

Correspondence Address: MORRISON & FOERSTER LLP, 755 PAGE MILL RD, PALO ALTO, CA, 94304-1018, US

(73) Assignee: Council of Scientific and Industrial Research, New Delhi (IN)

(21) Appl. No.: 10/528,443

(22) PCT Filed: Dec. 30, 2003
(86) PCT No.: PCT/IN03/00414

§ 371 Date: Feb. 22, 2006

(51) Int. Cl. G01N 33/53; C12Q 1/37

(52) U.S. Cl. 435/7.1; 435/23

(57) ABSTRACT

The present invention provides that arginine, a biologically compatible molecule that is known to bind to the peptide backbone and negatively charged side-chains, increases the chaperone-like activity of a-crystallin significantly. Arginine, interestingly, restores the activity of mutant protein to a considerable extent. The invention shows the effect of arginine on the structural changes of a-crystallin by circular dichroism, fluorescence and glycerol gradient sedimentation. The invention shows that arginine brings about subtle changes in the tertiary structure and significant changes in the quaternary structure of a-crystallin and enhances its chaperone activity significantly. This invention thus has role in designing strategies to improve chaperone function for therapeutic applications.

### FIELD OF INVENTION

The present invention provides that arginine, a biologically compatible molecule that is known to bind to the peptide backbone and negatively charged side-chains, increases the chaperone-like activity of α-crystalin significantly. Arginine, interstingly, restores the activity of mutant protein to a considerable extent. The invention shows the effect of arginine on the structural changes of α-crystallin by circular dichroism, fluorescence and glycerol gradient sedimnentation. The invention shows that arginine brings about subtle changes in the tertiary structure and significant changes in the quaternary structure of α-crystallin and enhances its chaperone activity significantly. This invention thus has role in designing strtegies to inprove chaperone function for therapeutic applications.

### BACKGROUND OF THE INVENTION

α-Crystallin, a multimeric protein of the eye lens, is made up of two homologous gene products, αA-, and αB-crystallins. αB-crystallin is expressed in significant levels in other tissues such as heart, kidney, brain, muscle, etc., while αA-crystallin is expressed in trace amounts in the spleen and thymus, (Bhat & Nagineni, 1989; Dubin et al., 1989; Iwaki et al., 1989; Kato et al., 1991). αB-Crystallin is stress-inducible and is expressed at elevated levels under certain disease conditions (Aoyama et al., 1993; Renkawek et al., 1994; Groenen et al., 1994). αA- and αB-crystallin share structural and sequence homology with small heat shock proteins (sHSPs) (Ingolia & Craig, 1982; Merck et al., 1993). α-Crystallin exhibits molecular chaperone-like activity in preventing the heat-induced aggregation of other proteins (Horwitz, 1992). It is now established that both αA- and αB-crystallin, either in homo-multimeric or hetero-multimeric states, exhibit molecular chaperone-like properties in preventing aggregation of other proteins (Sun et al., 1999; Datta & Rao, 1999), protecting the enzyme activity from heat- or other stress-induced inactivation (Hook & Harding, 1997; Hess & Fitzgerald, 1998; Marini et al., 2000; Rajaraman et al., 2001) and in a few cases, assisting the refolding (Rajaraman et al., 2001; Goenka et al., 2001; Rawat & Rao, 1998; Ganea & Harding, 2000). The inventors have earlier investigated the chaperone-like activity of α-crystallin towards photo-induced aggregation of γ-crystallin, DTT-induced aggregation of insulin and the refolding-induced aggregation and β-, and γ-crystallins and demonstrated that it is possible to enhance the chaperone-like activity of α-crystallin (see Reviews by Rao et al., 1998 & 2002 and the references therein). Our results showed that a structural perturbation by temperature in α-crystallin, involving increased exposure of hydrophobic surfaces, enhanced the chaperone-like activity of α-crystallin several-fold (reviews by Rao et al., 1998 & 2002). Subsequent studies from other laboratories (Das & Surewicz, 1995; Borkman et al., 1996; Palmisano et al., 1995) have shown similar results. Smith et al. (1996) found that hydrophobic regions around residues 32-37 and 72-75 in αA-crystallin and 28-34 in αB-crystallin become solvent-exposed above 30° C. Sharma et al. (1997 & 2000) identified specific target protein-binding regions, depending on the nature of the target proteins, that span the α-crystallin domain. Evidence has accumulated over the years that temperature regulates the chaperoning process in general through structural perturbation of the chaperone molecules such as Hsp70 (Craig & Gross, 1991; Leung et al., 1996) as well as small heat shock proteins (Rao et al. 1998; Gu et al., 2002; Haslbeck et al., 1999; van Montfort et al., 2001). Interestingly, the structurally perturbed state of Hsp 16.3 at low concentrations of guanidine hydrochloride (Gdn.HCl) has also been shown to exhibit enhanced chaperone-like activity (Yang et al., 1999).

In addition to temperature, it has been observed that low concentrations of the denaturant, urea, also enhance activity corroborating our hypothesis (reviews by Rao et al., 1998 & 2002). Gdn.HCl has been shown to enhance the chaperone-like activity of α-crystallin (Das & Liang, 1997). Loss or decreased chaperone function appears to be molecular basis for a growing number of diseases. If biologically compatible small molecules could be used to cause a structural perturbation and enhance the chaperone-like activity of α-crystallin, they would be of therapeutic significance. Therefore the inventors in the present study set out to investigate the effect of the biologically compatible small molecule, Arg.HCl, on the chaperone-like, anti-aggregating activity of α-crystallin. Guanidinium salts, including Arg.HCl, are known to bind to the peptide backbone as well as side chains of negatively charged amino acids and tryptophan (Arakawa & Timasheff, 1984; Lin & Timasheff, 1996; Timasheff & Arakawa, 1988).

The chaperone-like activity of α-crystallin in preventing aggregation of other proteins is particularly important in the context of the eye lens in maintaining its transparency. Our results show that, indeed, Arg.HCl can enhance the chaperone-like activity of bovine eye lens α-crystallin as well as the recombinant human αA- and αB-crystallins several-fold. More interestingly, it can also enhance activity of the mutant αB-crystallin (R120G-αB) that causes desmin-related myopathy and congenital cataract (Vicart et al., 1998), perhaps due to its decreased activity (Kumar et al., 1999). Our results with guanidinium compounds such as Arg.HCl and AGdn.HCl show that enhanced activity of α-crystallin might be due to altered tertiary and quaternary structure.

### OBJECTS OF THE INVENTION

The main object of the invention provides a method enhancing the molecular chaperone activity of α-crystallin by Argnine Hydrochloride

Another object of the invention provides increase in molecular chaperone activity of α-crystallin by binding of Argnine Hydrochloride to the negatively charges side chains of the α-crystallin

Still another object of the invention increase in molecular chaperone activity of α-crystallin by Argnine Hydrochloride by preventing aggregation of proteins

### SUMMARY OF THE INVENTION

The present study has shown that Arginin hydrochloride (Arg.HCl) induced increase in the chaperone activity is -more pronounced for αB-crystallin than for crystallin. Further a point mutation, R120G, in αB-crystallin is also restored by Arg.HCl to a considerable extent. Arg,HCl also brings about structural changes of α-crystallin by circular dichroism, fluorescence and glycerol gradient sedimentation. It has been shown that Far-UV CD spectra has no significant compared to near-UV CD spectra in bringing changes in secondary structure. The glycerol gradient sedimentation also shows a significant decrease in the size of α-crystallin oligomer in the presence of arginine. There is a increase in exposure of hydrophobic surfaces of α-crystallin in the presence of arginine. These results show that arginine brings about subtle changes in the tertiary structure and significant changes in the quaternary structure of α-crystallin and enhances its chaperone-like activity significantly. This study should prove useful in designing strategies to improve chaperone function for therapeutic applications.

### DETAIL DESCRIPTION OF ACCOMPANYING DRAWINGS/FIGURES

FIG. 1: Effect of Arg.HCl on the chaperone-like activity of calf eye lens α-crystallin towards the DTT-induced aggregation of insulin at 37° C. (A), aggregation of 0.2 mg/ml insulin in buffer alone (-O-); and in the presence of 0. mg/ml α- crystallin (-●-). (B) aggregation of insulin in buffer containing 200 Mm Arg.HCl (-Δ-), aggregation of insulin in the presence of α-crystallin (0.1 mg/ml) and 200 mM Arg.HCl (-▴-). Symbols represent different curve t)pes and not data points. Inset shows the change in percent protection offered by acrystac in as a function of Arg.HCl concentration.

FIG. 2: Effect of Arg.HCl on the chaperone-like activity of recombinant human αA- and αB-crystallins towards the DTT-induced aggregation of insulin at 37° C. Percent protection offered by 0. mg/ml αA-crystallin (0), and 0.05 mg/ml αB-crystallin (●), towards the aggregation of insulin (0.2 mg/ml) as a function of Arg.HCl concentration.

FIG. 3 Effect of Lys.HCl on on the chaperone-like activity of a-crystallin towards DTT-induced aggregation of insulin at 37° C. Insulin (0.2 mg/ml) aggregation in the absence (open symbols) and in the presence (closed symbols) of α-crystallin (0.1 mg/ml): 100 mM (circles) and 300 mM (triangles) Lys.HCl. Symbols represent different curve types and not data points.

FIG. 4: Effect of Arg.HCl (-O-), Gdn.HCl (-●-) and aminoguanidine hydrochloride (A) on the chaperone-like activity of calf eye lens α-crystallin towards the DTT-induced aggregation of insulin at 37° C. α-Crystallin and insulin were taken at concentrations of 0.1 and 0.2 mg/ml respectively.

FIG. 5: Effect of Arg.HCl on the chaperone-like activity of α-crystallin at 25° C. α-crystallin to insuin ratio of 1:1 (wtw).

FIG. 6: Solubilization of pyrene by a-crystallin as a function of concentration of Arg.HCl at 37° C. S_{α}=A_{α}−A_{b}, S_{αArg}=A_{αarg}−A_{bArg}, where A is the absorbance of the sample at 338 nm, S_{α} represents the solubility of pyrene by a-crystallin and S_{αArg} represents the solubility of pyrene by α-crystallin in the presence of Arg.HCl.

FIG. 7: Relative fluorescence intensity of ANS bound to α-crystallin in the presence of increasing concenh ions of Arg.HCl at 37° C.,

FIG. 8 Circular dichroism spectra of a-crystallin in the presence and in the absence of Arg HCl at 37° C. Panel A: Near-UV CD spectra; Panel B: Far-UV CD spector a-Crystallin in Buffer A alone (curve 1) and the buffer containing 100 mM (curve 2), 200 mM (curve 3) and 300 mM (curve 4) Arg.HCl. To record far UV-CD spectra, Dl-Arg.HCl was used. [θ]_{MRM} is mean residue mass elipticity.

FIG. 9: Sedimentation of the Arg.HCl-treated (-●-) and untreated (O) a-crystallin through a linear glycerol gradient (10-40%). The samples were incubated at 37° C. for 2 h before loading on to the gradient. The positions of proteins used for standard molecular masses are also indicated: a=aldolase (158 kDa), b=catalase (232 kDa) and c=thyroglobulin (669 kDa). See materials and methods for details.

FIG. 10: Effect of Arg.HCl on the chaperone activity of the mutant, R120GaB-crystallin towards the DTT-induced aggregation of insulin at 37° C. The percent protection represent-the percent prevention of aggregation of insulin by these protein as monitored by light scattering. The negative value of the percent protection indicates the extent of increase in the aggregation of the samiple copared to that of insuin alone.

### DETAIL DESCRIPTION OF THE INVENTION

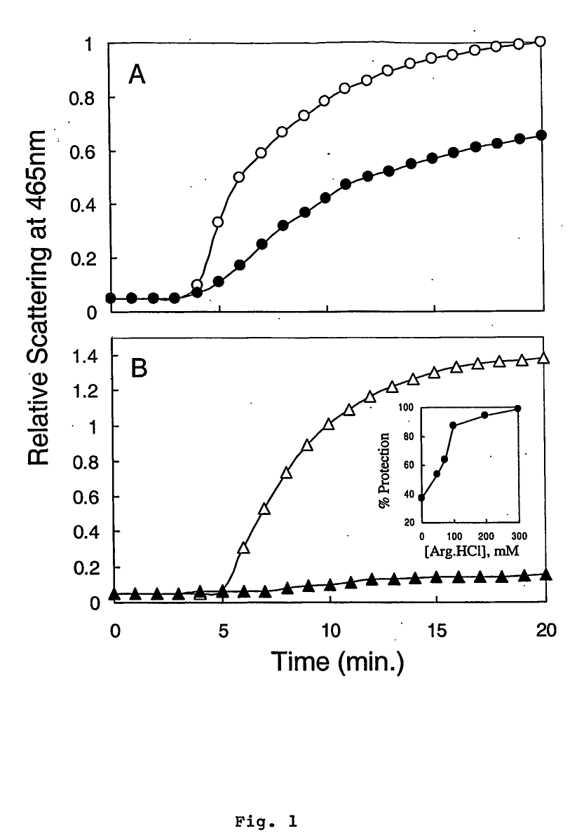

Structural perturbation of α-crystallin leads to several fold increase in its chaperone activity (24-27,31,32). In order to find out whether some biologically compatible compounds can perturb the structure and enhance the chaperone activity of a crystallin, guanidinium compounds such as hydrochlorides of arginine, aminoguanidine and guanidine have been investigated. Arginine is known to interact with the peptide bonds of proteins as well as negatively charged side chains of amino acids (37,38). In present invention the effect of Arg.HCl on the chaperone-like activity of acrystallin has been proved. DTT-induced aggregation of inslin in the absence or in the presence of α-crystallin at 37° C. and the effect of varying concentrations of Arg.HCl are shown in FIG. 1. α-Crystallin prevented the aggregation of insulin partially (˜36% prevention of aggregation) at a 1:2 (w/w) ratio of a-crystallin:insulin (FIG. 1A). FIG. 1B shows that at the same weight ratio, a-crystallin prevents the aggregation of insulin completely in the presence of 200 mM Arg.HCl. Arg.HCl alone does not prevent the aggregation of insulin; on the contrary, we observed that it leads to its significantly increased aggregation. Despite this effect of Arg.HCl on the aggregation of insulin, α-crystallin in the presence of Arg.HCl showed complete prevention of aggregation. This result. shows that Arg.HCl increases the chaperone-like activity of α-crystlin significantly. The inset in FIG. 1B shows that the observed increase in the activity is dependent on Arg.HCl concentration, indicating a dose-dependent interaction of Arg.HCl with α-crystallin.

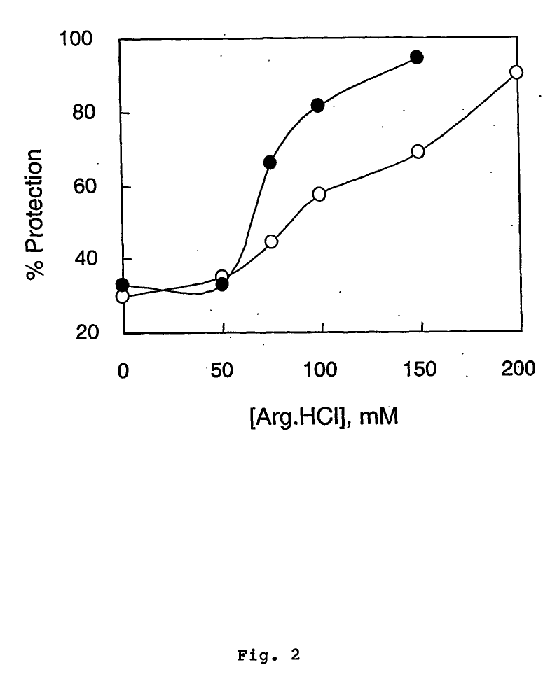

Eye lens α-crystallin is a heteromultimeric protein composed of two types of subunits—α- and αB-crystallin—with the subunit ratio varying from species to species (44). Both and αB-crystallin form homomultimers; however, they exhibit differences in their chaperone-like activities as well as structural stability (14,15,45). In order to investigate the possible differences in the Arg.HCl-induced increase in the chaperone activity, we sudied the effect of Arg.HCl on the activities of recombinant human α-A- and αB-crystallin towards the DTT-induced aggregation of insulin. In conformity with earlier reports (14,15,45), we found that αB-crystallin was more effective in preventing the aggregation of insulin than αA-crystallin; it almost completely prevented the aggregation of insulin at a 1:2 (w/w) ratio of aB rystallin to insulin. To see the effect of Arg.HCl onr their chaperone-like activities, we used a 1:2 ratio (w/w) for α-crystallin to insulin; 1:4 (w/w) ratio of α-crystallin to inslin since αB-crystallin is more effective as a chaperone. FIG. 2 shows that Arg.HCl enhanced the activity of both αA- and αB-crystallin, the increase being much sharper for αB-rystallin than for αA-crystallin. The Arg.HCl-induced enhancement of the activity of αB-crystallin is more pronounced than that of α-crystallin.

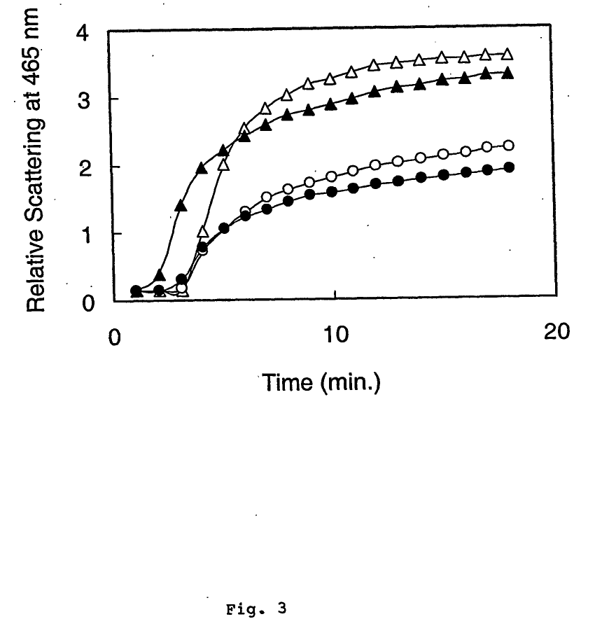

Lysine hydrochloride, another basic amino acid is also known to interact with peptide bonds of proteins as well as negatively charged amino acids (38). We, therefore, tested whether Lys.HCl also can increase the chaperone activity of α-crystallin. However, in the present study unlike Arg.HCl, Lys.HCl did not enhance the chaperone-like activity of α-crystallin towards the DTT-induced aggregation of insulin (FIG. 3). In fact, it reduced the percent protection offered by α-crystallin from 36% in the absence of Lys.HCl to 17% in the presence of 100 mM Lys.HCl. At a concentration of 300 mM Lys.HCl, the percent protection further reduced to about 8%. This shows that the effect of Arg.HCl is specifically due to the presence ofthe guanidinium group present in its side chain.

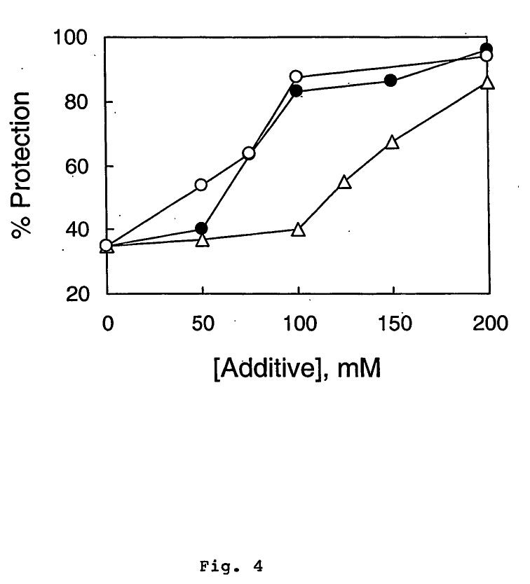

Incubating α-crystallin with low concentrations of Gdn.HCl (0.8-1.0 M) increases the exposure of its hydrophobic surfaces with an accompanying enhancement in its chaperone-like activity (35). Recently, the chaperone-like activity of a small heat shock protein, Hsp 16.3, from Mycobacterium tuberculosis has also been shown to be enhanced due to Gdn.HCl-induced perturbation of its structure (46). Further, a recent study by Inomata et al. (47) showed that aminoguanidine, when fed orally to Shumiya cataract rats—a hereditary cataract model in which lens opacity appears spontaneously in the nuclear and perinuclear portions at 11-12 weeks of age—inhibited the opacification of the lens. Aminoguanidine was also found to inhibit cataract formation in diabetic rats (48). In the present study a comparison between the effect of Arg.HCl, Gdn.HCl and aminogumidine hydrochloride on the chaperone-like activity of α-crystallin was undertaken. FIG. 4 shows that Arg.HCl and Gdn.HCl are comparable in their ability to enhance the activity of α-crystallin. Aminoguanidine hydrochloride also enhanced the activity of α-crystallin, but to a lesser extent (FIG. 4).

In order to find out whether Arg.HCl-induced increase in chaperone activity of α-crystallin is,.also observed with other protein aggregation systems, in the present study it has been investigated the effect of α-crystallin incubated with Arg.HCl on the thermally induced aggregation of ξ-crystallin aggregates at 43° C. (49). α-crystallin, at a 1:4 (w/w) ratio of α- to ξ-crystallin, prevented the aggregation of ξ-crystallin to the extent of 53%. Upon preincubation with 100 mM Arg.HCl, the protective ability of α-crystallin increased to ˜81%. Similar Arg.HCl-induced enhancement of the chaperone-like activity of α-crystallin is observed towards DTT-induced aggregation of a-lactalbumin at pH 6.5 (data not shown).

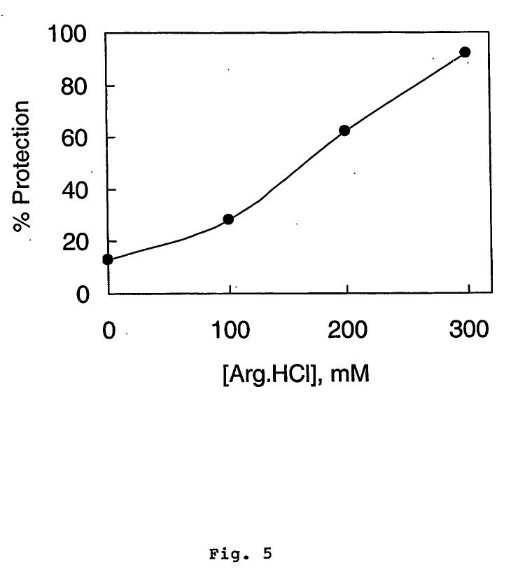

The investigators have earlier shown that the chaperone-like activity of α-crystallin is increased several-fold at temperatures beyond 30° C. In order to find out if arginine can mimic the effect of elevated temperature in enhancing the chaperone-like activity of α-crystallin, the effect of Arg.HCl on the chaperone-like activity of α-crystallin at 25° C. (FIG. 5) was investigated. At a α-crystallin to insulin ratio of 1:1 (w/w), the protection offered by α-crystallin is approximately 13%. In the presence of 100 mM Arg.HCl, the protection increased to 28%; as the concentration of Arg.HCl increased, the chaperone-like activity increased and at 300 mM Arg.HCl, it almost completely prevented the aggregation of insulin. Aminoguanidine hydrochloride also showed a similar effect on the activity of α-crystallin at 25° C., but to a lesser extent.

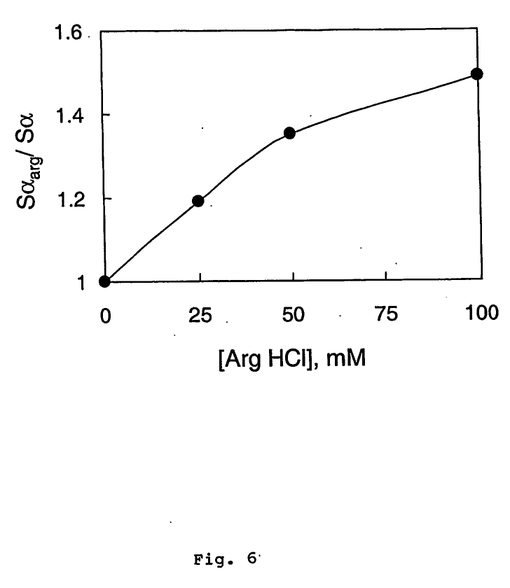

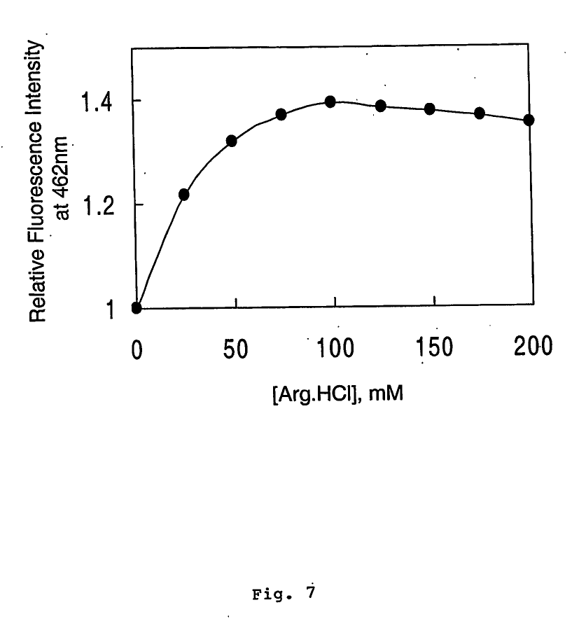

Earlier studies (24-26, 28-31) have shown that structural perturbation of α-crystallin leads to increased exposure of hydrophobic surface. Hydrophobic interactions play a crucial role in the recognition between chaperones and partially unfolded proteins. Since α-crystallin exhibits enhanced chaperone-like activity in the presence of Arg.HCl, the accessibility of the hydrophobic surfaces of α-crystallin as a function of Arg.HCl concentration by solubilizing the hydrophobic fluorophore, pyrene was investigated. Pyrene is sparingly soluble in water. In the presence of α-crystallin (0.3 mg/ml), there is a 4.2 fold increase in the solubility of pyrene at 37° C. In the concentration range studied, Arg.HCl alone does not increase the solubility of pyrene significantly. However, pyrene-solubilization by α-crystalhin is found to be significantly more in the presence of Arg.HCl than in its absence. The α-crystalin-solubilized fraction of pywne (See Examples) increased progressively with increase in Arg.HCl concentration (FIG. 6). These results suggest that there is an increase in the exposure of hydrophobic surfaces of α-crystallin in the presence of Arg.HCl. This aspect was further investigated using ANS, a fluorophore that binds to and reports on the hydrophobic surfie of a protein (50,51). In the presence of Arg.HCl, the binding of ANS to α-crystallin increased till the concentration of Arg.HCl reached 100 mM (FIG. 7). At this concentration of Arg.HCl, there was ˜38% increase in the fluorescence intensity of ANS at the emission maximum. Beyond this concention of Arg.HCl, there was a marginal drop in the fluorescence intensity at the emission naximum (FIG. 7). The pyrene-solubilization studies as well as the ANS-binding studies show that Arg.HCl increases the accessible hydrophobic surfaces of a-crystallin. We further studied structural changes in a-crystallin using intrinsic fluorescence, near- and far-UV CD spectroscopy and glycerol density gradient centrifugation.

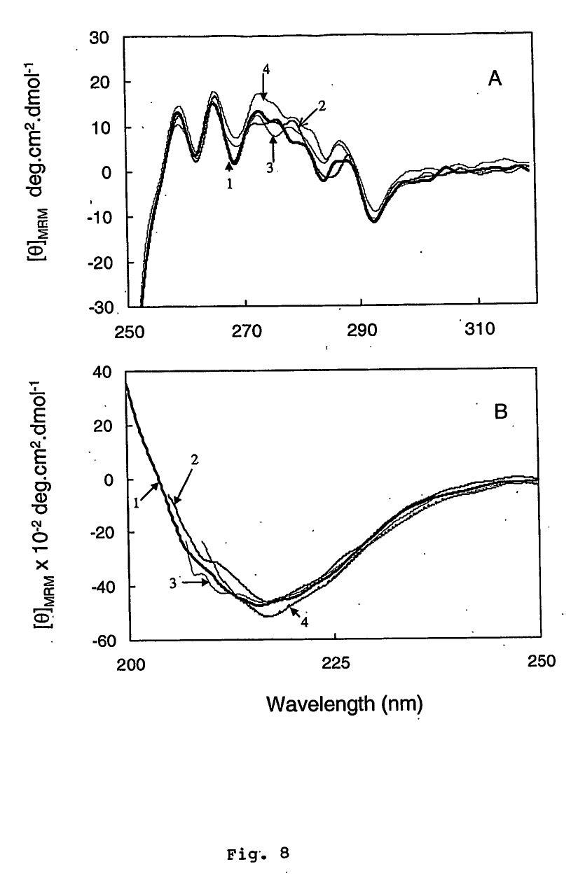

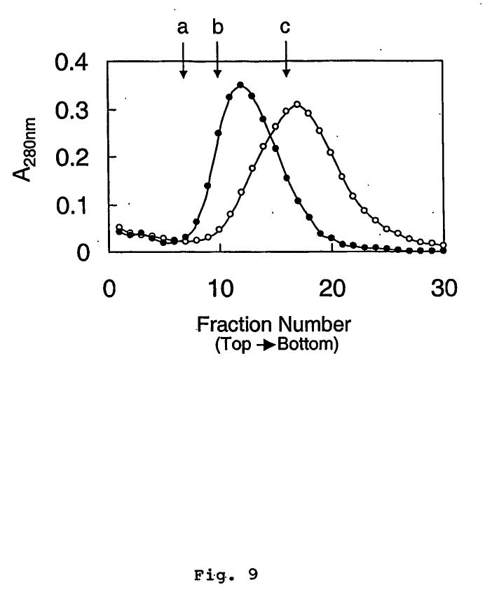

The intrinsic tryptophan fluorescence spectrum of α-crystallin did not change considerably in the presence of Arg.HCl, suggesting that there is no significant change in the tryptophan environment. The near-UV CD spectra of α-crystallin in the absence and presence of various concentrations of Arg.HCl are shown in FIG. 8A. The CD profiles of α-crystallin with increasing concentrations of Arg.HCl do not overlap. The profile in the region of 270-290 nm shows some differences suggesting subtle conformational changes at the tertiary structural level. Since L-Arg.HCl gives a strong signal in the far-UV region below 230 nm experiments using DL-Arg.HCl to perforn-far-UV CD studies were undertaken. The far-UV CD spectra of α-crystallin in the absence and the presence of various concentrations of Arg.HCl arc shown in FIG. 8B. The far-UV CD spectra almost overlap, with a minor change in the lower wavelength region. The minima, however, remain unchanged suggesting that the secondary structure, largely beta-sheet structure, of α-crystallin is not altered in the presence DL-Arg.HCl. In order to assess changes in the quaternary structure of the protein, sedimentation experiment through a 10-40% glycerol gradient in the absence and the presence of 300 mM Arg.HCl were performed FIG. 9 shows significant decrease in the size of α-crystallin in in the presence of Arg.HCl. We also used aldolase, catalase and thyroglobulin as molecular mass standards and estimated the molecular mass of α-crystallin. These results show that molecular mass of α-crystallin, which is estimated to be ˜700 kDa in the absence of Arg.HCl, is significantly decreased in the presence of Arg.HCl (˜360 kDa). The half-width of the profile of α-crystalibn in the presence of Arg.HCl is significantly less (6.25 x-axis units) than that in its absence (8.75 x-axis units), indicating that the polydispersity of the protein is also decreased in the presence of Arg.HCl. It was also observed similar decease in the size as well as the half-width of the profiles of α-A- and α-B crystallin (data not shown). Thus, these results show that Arg.HCl can bring about subtle changes in the tertiary structure and significant changes in the quaternary structure of either the homo-multimers or the hetero-multimere of α-A- and α-B crystallin. Such changes also lead to increased chaperone activity of α-crystallin.

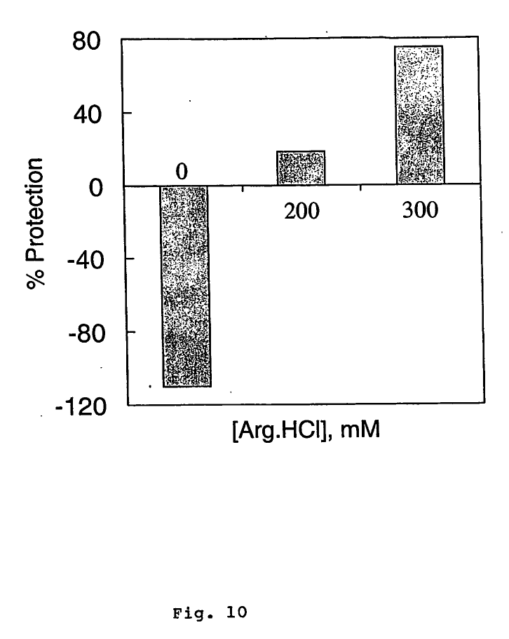

Mutations in α-A- and α-B-crystallin at the conserved argjnine residues 116 and 120 respectively, are known to affect structure and chaperone-lke activity in vitro (40, 52-54). These mutations are known to result in congenital cataract and desmin-related myopalhy (55,39). It would be of interest to investigate in this context whether the additive, ArJKI, can restore/increase the chaperone-like activity of these mutants. FIG. 10 shows the effect of Arg-HCl on the chaperone activity of R120G αB-crystallin towards the DTT-induced aggregation of insulin at 37° C. At a 0.5:1 (w/v) ratio of R120G α-crystallin to insulin, the light scattering of the sample is significantly more as compared to that of insulin in the absence of the mutant crystallin, indicating that it does not offer protection to insulin. The apparent increase in light scattering compared to the control is shown as a negative percentage protection in the figure (FIG. 10). Interestingly, increasing concentrations of the additive Arg.HCl increase the protective effect of R120G α-crystallin (FIG. 10), the extent of protection being ˜75% at 300 mM Arg.HCl. However, αA-crystallin mutant R116C did not show similar sensitivity to Arg.HCl. Even 500 mM did not enhance the activity of R116C αA-crystaIlin (data not shown). Earlier studies by the inventors have shown that R12OG oBcrystalBin assembles into multimers with slightly increased molecular mass compared to the wild-type αB-crystallin R116C α-A-crystallin, on the other hand, forms very large (800-2000 kDa), polydisperse, multimers (40). It is not clear whether such difference in the multimeric nature of these mutants in some way leads to the observed differences in the ArHCl-induced effects on the chaperone activity. As mentioned earlier, FIG. 2 shows that the extent of Arg.HCl-induced increase in the chaperone activity of αA-crystallin is lesser than that of αB-crystallin. Therefore, these results suggest that the wild type and mutant αA-crystallin, in general, are less sensitive towards Arg.HCl-induced changes in structure and fineiorL

Taken together, the present invention shows that in the presence of Arg.HCl, α-crystallin undergoes subtle tertiary structural changes and significant quaternary structural changes accompanied by increase in the accessible hydrophobic surfaces of the protein. These strutural changes result in a significant enhancement in the chaperone-like activity of α-crystallin. The fact that lysine hydrochloride could not enhance the chaperone activity of α-crystallin whereas Arg.HCl and other guanidinium compounds such as guanidine hydrochloride and aminoguanidine hydrochloride could markedly increase its chaperone activity shows selective interaction of the guanidinium group in arginine with α-crystallin. It is also important to note that guanidine hydrochloride and atinoguanidine hydrochloride also enhanced the chaperone activity of α-crystallin, however, to a lesser extent compared to Arg HCl. It was also observed a change in the near-UV CD spectrum and size of aystallin in the presence of Lys.HCl (data not shown) indicating that Lys.HCl can also interact with the protein. However, it failed to increase the chaperone-function of α-crystallin; rather it decreased the activity. Thus, it appears that the mode of interaction of Lys.HCl with α-crystallin is either distinctly different from that of the guanidinium compounds such as Arg.HCl and aminoguanidine HCl or the α-crystallin-target protein complex is unstable and aggregation-prone in the presence of Lys.HCl. It is also known that Arg.HCl and Lys.HCl exhibit opposite effects on the stability of globular proteins (37) though both these amino acids can interact with peptide groups and negatively charged side chains ofproteins (36-38).

Growing evidences suggest that some small molecules modulate chaperone function in a specific manner. Osmolytes such as glycinebetaine, proline and glycerol have been shown to activate the molecular chaperones, GroEL and D and ClpB (56). The cellular metbolite, pantethine has been shown to increase the anti-aggregation activity of allin (57). The present invention shows that Arg.HCl, a biologically compatible amino acid, can significantly improve the chaperoning function of α-crystallin by specific interactions with the protein. Present invention also demonstrates that arinoguanidine, which has been earlier shown to inhibit lens opacification in anima models (47,48), also increases the chaperone activity of α-crystallin. This result suggests that the enhancement of chaperone activity of C-crystallin is one of the probable mecanisms, if not the sole mechanism, involved in the inhibition of lens opacification by aminogumidine in some animal models. Arg.HCl might confer beneficial effects against lens opacification since, as our results show, it is more effective in increasing the chaperone activity of α-crystallinthan aminoguanidine. The inventors are currently testing this possibility in animal models. In addition, Arg-HCl may offer other beneficial effects, as it is the substrate for enzymatic production of nitric oxide and believed to confer cardiovascular protection (58-60), and exhibits antioxidant properties in scavenging superoxide radical (61). Thus, Arg.HCl is one of the promising agents to exhibit pleotropic beneficial effects in general health and disease reisace. Accordingly, the main embodiment of the present invention relates to a method for enhancing molecular chaperone activity of α-crystallin (comprising of forms αA-crystalline and αB-crystalline) with a biological compatible amino acid molecule of Arginine Hydrochloride, said method comprising the steps of:

(a) isolating and purifying α-crystallin from calf eye lenses by convention methods (as described in reference 24), and

(b) reacting α-crystalline in the presence of phosphate buffer of pH 7.4 with Arg.HCl and aggregation systems selected from group of insulin or α-crystalline in presence or absence of DTT, and

(c) determining the enhancement in chaperone activity of α-crystallin in presence of Arg.HCl by fluorescence spectrophotometer.

Another embodiment of the present invention relates to the Arginine hydrochloride (Arg.HCl) wherein the Arg.HCl binds to the peptide backbone and negatively charged side chains of α-crystalline to enhance chaperone activity.

Still another embodiment of the present invention relates to the Arg.HCl wherein Arg.HCl is in the range of about 50 to 350 mM. Yet another embodiment of the present invention relates to the Arg.HCl wherein Arg.HCl is in the range of about 100 to 300 mM. One more embodimentof the present invention relates to the Arg.HCl wherein Arg.HCl enhances the chaperone activity of α-crystalline by about 95%. Another embodiment of the present invention relates to the Arg.HCl wherein Arg.HCl enhances the chaperone activity of α-crystalline by about 90%. Still another embodiment of the present invention relates to the Arg.HCl wherein the Arg.HCl enhances the chaperone activity of α-crystalline by about 90% in presence of various aggregation systems.

Yet another embodiment of the present invention relates to the Arg.HCl wherein Arg.HCl enhance the chaperone activity of α-crystalline by about 81% in presence of various aggregation systems.

One more embodiment of the present invention relates to the aggregation systems wherein the aggregation systems are selected from group comprising of insulin, α-crystallin and related compounds.

Another embodiment of the present invention relates to the Arg.HCl wherein Arg.HCl at a temperature of about 30° C. protects the ac-crystalline by about 35%. Still another embodiment of the present invention relates to the Arg.HCl wherein Arg.HCl at a temperature of about 30° C. protects the α-crystalline by about 28%. Yet another embodiment of the present invention relates to the Arg.HCl wherein Arg.HCl brings about subtle changes in the tertialry structure and significant changes in the quatemary structure of both homo-multimers or hetero-multimers of αA-crystalline and αB-crystalline to enhance the chaperone activity. One more embodiment of the present invention relates to the Arg.HCl wherein presence Arg.HCl the molecular mass of α-crystallinie is reduced −360 kDa thereby bringing about subtle changes in the tertialry structure and significant changes in the quaternary structure of both homo-multimers or hetero-multimers of αA-crystalline and αB-crystalline to enhance the chaperone activity.

Another embodiment of the present invention relates to the wild type and mutant αA-crystalline wherein wild type and mutant xα-crystalline are less sensitive to Arg.HCl than αB-crystalline, thereby enhancing the chaperone activity.

Yet another embodiment of the present invention relates to the protection of mutant αB-crystalline (R120αB-crystallin) wherein protection of mutant αB-crystalline (R120αB-crystallin) is about 80% in presence of Arg.HCl.

Still another embodiment of the present invention relates to the protection of mutant αB-crystalline (R120αB-crystallin) wherein the protection of mutant αB-crystalline (R120αB-crystallin) is about 75% in presence of Arg.HCl.

One more embodiment of the present invention relates to the Arg.HCl wherein Arg.HCl enhances the α-crystalline chaperone activity by about 45% by exposing the hydrophobic surfaces of α-crystalline.

Still another embodiment of the present invention relates to the Arg.HCl wherein Arg.HCl enhances the α-crystalline chaperone activity by about 38% by exposing the hydrophobic surfaces of α-crystalline.

The following examples are given by way of illustration of the present invention and therefore should not be construed to limit the scope of the present invention.

### EXAMPLES

### Example 1

Arginine hydrochloride (Arg.HCl), DLarginine hydrochloride ([)L-Arg.HCl), aminoguanidine hydrochloride. insulin and pyrene were obtained from Sigma Chemical Company, USA 8-Anilinonaphthalene-1-siilphonic acid (ANS) was obtained from Aldrich Chemical Company, USA. Guanidine hydrochlorlde was purchased from Serva, Germany All other chemicals used in the studies were of Analytical Reagent grade. In all the experiments Larginine hydrochloride, hereafter referred to as Arg.HCl. was used except to record far-UV circular dichrnism spectra, where DLArg.HCl was used

### Example 2

Preparation of α-crystallin and ε-crystallin

α-Crystallin was purified from calf eye lenses previously described (24). Recomhinant human α-A- and α-B-crystallins were prepared by cloning, overexpression in Eschenciia coli and purification as described earlier (40). ε-crystallinwas purified from guiinea pig eye lenses as described bv Rao and Zigler (41).

### Example 3

Assays for chaperone-like activity pf α-crystallin.

The DTT-induced aggregation of insulin and the effect of α-crystallin were studied as described earlier (25,42). All chaperone assays were carried out in 10 mM phosphate buffer. pH 7.4, containing 100 mM NaCl (Buffer A). The buffer alone or the buffer containing α-crystallin (0.1 mg/ml), α-A-crystallin (0.1 mg/ml) or α-B-crystallin (0.025 mg/ml) and the required amount of Arg.HCl was equilibrated at 37° C., for 3 minutes with constant stirring in the sample holder using a Juiabo thermostated water bath. Insulin (0.2 mg/ml) was then added and reduction of insulin was initiated by adding 20 ρL of 1 M DTT to 1.2 ml of the sample. The extent of aggregation of insulin was measured as a function of time by monitoring 90° scattering at 465 nm using a Hitachi F-4000 fluorescence spectrophotometer. The excitation and emission band passes were set at 3 nm.

Chaperone activity measurement of α-crystallin towards the thermal aggregation of ε-crystallin was performed as described in our earlier study (25). Buffer A either in the absence or in the presence of α-crystallin (0.025 mg/ml) and required concentrations of Arg.HCl was incubated at 43° C. for 3 min in the thermostated cuvette holder of the fluorescence spectrophotometer. ε-Crystallin was then added to a final concentration of 0.1 mg/mL The extent of aggregation was monitored as a function of time by measring the scalr of 465 nm light as descnbed above,

### Example 4

Solubilization of Pyrene

α-Crystallin solution (0.3 mg/ml in Buffer A) in the absence or the presence of varying concentrations of Arg.HCl was stirred with mM pyrene suspension at 37° C. for 30 minutes. The mixtre was centrifuged at 14,000 rpm for 5 minutes to remove the unsolubilized pyrene. Optical density of the supernatant was measured at 338 nm in a Hitachi-330 TV-Visible spectrophotometer. Solubility of pyrene in Arg.HCl solutions alone was also measured using the above procedure. The solubilization of pyrene by α-crystaain alone or by α-crystallin in the presence of varying concentrations of Arg.HCl was obtained after conrecting for the appropriate blanks. In order to assess the extent of increase in pyrene solubilization by α-crstallin in the presence of Arg.HCl, the ratio of pyrene solubilized by a in the presence of given Arg.HCl concentration (Soarg) to pyrene solubilized by a α-crystallin alone (S_{α}) was plotted as a function of Arg.HCl eoncenotion

### Example 5

Circular Dichroism Studies

Circular dichroism spectra were recorded using a Jasco J-715 Spectropolarimeter. All spectra are the average of 4 accumulations. Far- and near-UV CD spectra of α-crystallin (1.5 mg/nl) in the absence and the presence of various concentrations of Arg.HCl were recorded at 37° C. using themostated 0.01 cm and cm pathlength cuvettes respectively All spectra were corrected for the respective blanks Since L-arginine liydochloride shows a large CD signal below 230 nm we used DL-Arg.HCl to record the far-UV)D spectra of α-crystallin.

### Example 6

Fluorescence Studies

Fluorescence spectra were recorded using a Hitachi F-4000 Fluorescence Spectrophotometerequipped with a thermostated cuvette holder. All the studies were performed at 37° C. All spectra were recorded in the corrected spectrum mode. The nirinsic tryptophan fluorescence spectra of α-crystllin (0.2 mg/ml) in Buffer A alone or in the buffer containing the required concentrations of Arg.HCl were recorded by exciting the sample at 295 nm with the excitation and emission band passes set at 3 nm.

To study the binding of the hydrophobic probe, ANS, to α-crystallin, 12 uL of 10 mM methanolic solution of ANS was added to 1.2 ml of a 0.5 mg/ml solution of α-crystallin in Buffer A alone or in the buffer containing the required concentrations of Arg.HCl. The samples were excited at 365 nm and emission spectra recorded from 400-600 nm at 37° C. The excitation and emission band passes were set at 3 and 5 nm, respectively. All spectra were corrected for the respective blanks. Spectra were recorded in the corrected spectrum mode.

### Example 7

Glycerol Gradient Centrifugation:

Glycerol gradient centrifugation was carried out essentially as described by Lambert et al. (43). α-Crystallin, α-A- or α-B-crystallin (2 mg/ml) was incubated in 50 mM Tris HCl buffer (pH 7.4) containing 100 mM NaCl, 1 mM EDTA alone or in the presence of 300 mM Arg.HCl for 2 hours at 37° C. The samples (0.5 ml) were loaded on top of a 12 ml linear grddient of glycerol (10-40%) made in 50 mM Tris-HCl buffer, pH 7.4, containing 100 mM NaCl and 1 mM EDTA. Arg.HCl-treated α-crystllin samples were run in the gradient also containing 300 mM Arg.HCl. The tubes were centriflged for 18 hours at 30000 rpm in a Beckman SW41 rotor at 4° C. Fractions (0.3 ml) were withdrawn from the top using a Haake-Buchler Auto Densi-Flowt HC gradient former/remover and optical density at 280 nm of the fractions were measured using a Shimadzu W-1601 spectrophotometer. To estimate the molecular masses of the α-crystallin samples, proteins with defined molecular masses such as thyroglobulin (669 kDa), catalase (240 kDa) and aldolase (158 kDa) were used.

### REFERENCES:

1. Bhat, S. P., and Nagineni, C. N. (1989) Biochem. Biophys. Res. Commun 158, 319-325

2. Dubin, R A, Wawrousek, E. F., and Piatigorsky, J. (1989) Mot. Cell. Biol. 9, 1083-1091

3. Iwaki J., Kume-Ikaki. A., Liem, R. K. H., and Goldman, J. E. 989) Cell 57, 71-784.

4. Kato, K., Shinohara, H., Kurobe, N., Goto. S. Inaguma, Y and Ohshima, K (1991 Biochim. Biophys. Acta 1080, 173-180

5. Aoyama, A, Steiger, R. H., Frohli, E., Schafer, R., VonDeimling, A., Wiestler, O. D. M., and Klemenz, R. (1993 ) Int. J. Cancer 55, 760-764

6. Renkawek, K., Voorter, C. E. M., Bosman, G. J. C. G. M., van Workum, F. P. A., and de Jong, W. W. (1994) Acta Neuropathol. 87,155-160

7. Groenen, P. J. T. A., Merck, K. B., de Jong, W. W., and Bloemendal, H. (1994) Eur. J Biochem. 225,1-19

8. Klemenz, R., Frohli, E., Steiger, R. H., Schafer, R., and Aoyama, A. (1991) Proc. Natl. Acad. Sci. U. S. A. 88, 3652-3656

9. Dasgupta, S., Homnan, T. C., Carper, D. (1992) Exp. Eye Res. 54, 461-470

10. de Jong, W. W Leunissen, J. A. M., Leenen, P. J. M., Zweers, A, and Versteeg, M. (1988) J. Biol. Chem. 263,5141-51491.

11. Ingolia, T. D., and Craig, E. A. (1982) Proc. Natl. Acad Sci. U.S.A. 79, 2360-2364

12. Merck, K. B., Gronen, P. J. T. A, Vooeter, C. E. M. de Haard-Hockman, W. A. Horwitz, J., Bloemendal, H., and de Jong, W. W. (1993) J. Biol. Chem. 268, 1046-1052

13. Horwitz, J. 1992) Proc. Natl. Acad. Sci. U.S.A. 89, 10449-10453

14. Sun, T. X,. Das, B. K., and Liang, J. J. N. (1999; J. Biol. Chem. 272,6220-6225

15. Datta, S. A., and Rao, C. M. (1999) 7. Biol. Chem, 274,34773-34778

16. Hook D. W. A., and Harding J. J. (1997) Eur. J. Biochem. 247, 380-385

17. Hess, J. and Fitzgerald, P. C. (1998 ) Mol. Vis. 4,29-32

18. Marini, I., Moschini, R., Del Corso, A., and Mura, U. (2000) J. Biol. Chem. 275, 32559-32565

19. Blakytny, R., and Harding, J. J. (1997) Exp. Eye Res. 64, 1051-1058

20. Rajaraman, K., Raman, B., Ramakrishna, T., and Rao, C. M. (2001) FEBSLett. 497, 118-123

21. Goenka, S., Raman, B., Ramakrishna, T., and Rao, C. M. (2001) Biochem. J. 359, 547-556

22. Rawat, U., and Rao, M. (1998) J. Biol. Chem. 273,9415-9423

23. Ganea, E., and Harding, J. J. (2000) Eur. J. Biochem. 345, 467-472

24. Raman, B., Ramakrishna, T., and Rao, C. M. (1995) J. Biol. Chem. 269,27264-27268

25. Raman, B., Ramakrishna, T., and Rao, C. M. (1995) FEBSLett. 365,133-136

26. Raman, B., Ramakrishna, T., and Rao, C. M. (1995) J. Biol. Chem. 270,19888-19892

27. Raman, B., and Rao, C. M. (1997) J. Biol. Chem. 272,23559-23564

28. Rao, C. M., Raman, B., Ramakrishna, T., Rajaraman, K., Ghosh, D., Datta, S., Trivedi, V. D., and Sukhaswami, M. B. (1998) Int. J. Biol. Macromol. 22,271-281

29. Palmisano, D. V., Groth-Vasselli, B., Farnsworth, P. N. and Reddy, M. L. (1995) Biochim. Biophys. Acta 1246,91-97

30. Borkman, R. F., Knight, G., Obi, B. (1996) Exp. Eye Res. 62, 141-148

31. Das, K. P., and Surewicz, W. K. (1995) FEES Lett. 369, 321-325

32. Smith, J. B., Liu, Y., and Smith, D. L. (1996) Exp. Eye Res. 63,125-128

33. Sharma, K. K., Kumar, R. S., Kumar, G. S., Quinn, P. T. (2000) J. Biol. Chem. 275, 3767-3771

34. Sharma, K. K., Kaur, R, and Kester, K. (1997) T Biochem. Biophys. Res. Commun. 239, 217-222

35. Das, B. K., and Liang, J. J-N. (1997) Biochem. Biophys. Res. Commun. 236,370-374

36. Arakawa, T., and Timnsheff, S. N. (1984) Biochemistry 23, 5924-5929

37. Lin, T-Y and Timasheff, S. N. (1996) Prot. Sci. 5, 372-38

38. Timasheff, S. N and Arakawa, T. (1988) in Protein Structure. A Practical Approach (Creighton, E. Ed.,) IRL Press, Oxford, pp 33 344

39. Vicart, P., Carron, A., Guicheney, P., Li, Z., Prevost, M., Faure, A., Chateau, D., ˆhapon, F., Tome, F., Dupert, J., Paulin, D., and Fardeau, M. (1998) Natun Genetics 20, 92-95

40. Kumar, I. V. S., Ramakrishna, , and Rao, C. M. (1999) J. Biol. Chem. 274, 24137-24141

41. Rao, P. V., and Zigler, Jr., J. S. (1992) Exp. Eye Res. 54, 627-630

42. Farahbaksh, Z Huang, Q. L., Ding, L. L., Altenbach, C Steinhoff, H., Horwitz, J. and Hubbell, W. L. (1995) Biochemistry 34, 509-516

43. Lambert, H., Charette, S. J., Bemier, A. F., Guimond, A., and Landry, J. (1999) J. Biol. Chem. 274,9378-9385

44. Siezen, R. J., Bindels, J. G., and Hoenders, H. J. (1978) Eur. J. Biochem. 91, 387-396

45. Reddy, G. B., Das, K. P., Petrash, J. M., and Surewicz, W. K. (2000) J. Biol. Chem. 275 4565-4570

46. Yang, H., Huang, S., Dai, H., Gong, Y., Zheng, C., and Chang, Z (1999) Prot. Sci. 8, 74-179

47. Inomata, M., Hayashi, M., Shumiya, S., Kawashima, S., and Ito, Y. (2000) J. Biochem. (Tokyo) 128,771-776

48. Swaimy, M. S., Green, K., and Abraham, E. C. (1996) Exp. Eye Res. 62, 505-510

49. Rao, P. V., Horwitz, J, and Zigler, Jr., J. S. (1994) J. Biol. Chem. 269,27264-27268

50. Stryer, L, S. (1965) J. Mol. Biol. 13, 482-495

51. Cardamone, M., and Puri, N. K. (1992) Biochem. J. 282, 589-593

52. Bova, M. P., Yaron, O., Huang, Q. L., Ding, L. L., Haley, D. A., Stewart, P. L., and Horwitz, J. (1999) Proc. Natl. Acad. Sci. USA 96,6137-6142

53. Pemg, M. D., Muchowski, P. J., van den Ijssel, P., Wu, G. J., Hutcheson, A. M., Clark, J. I., and Quinlan, R. A. (1999) J. Biol. Chem. 274,33235-33243

54. Shroff, N. P., Cherian-Shaw, M., Bera, S., and Abraham, E. C. (2000) Biochemistry 39,1420-1426

55. Litt, M., Kramer, P., Dante, M., Morticella, M., Muphy, W., Lovrien, E., and Welber, R. (1998) Human Mol. Genet. 7,471-474

56. Diamant, S., Eliahu, N., Rosenthal, D., and Goloubinoff, P. (2001) J. Biol. Chem. 276,39586-39591

57. Clark, J. I., and Huang, Q-L. (1996) Proc. Natl. Acad. Sci. U.S.A. 93,15185-15189

58. Suematsu, Y., Ohtsuka, T., Hirata, Y., Maeda, K., Imanaka, K., and Takamoto, S. (2001) Eur. J. Cardiothorac. Surg. 19,873-879

59. Shiono, N., 'Rao, V., Weisel, R. D., Kawasaki, M., Li, R. K., Mickle, D. A., Fedak, P. W., Tumiati, L. C., Ko, L., and Verna, S. (2002) Am. J. Physiol. Heart Circ. Physiol. 282, H805-815

60. Padilla, F., Garci-Dorado, D., Agullo, L., Inserte, J., Paniagua, A., Mirabet, S., Barrabes, J. A., Ruiz-Meana, M., and Soler-Soler, J. (2000) Cardiovasc. Res. 46, 412-420

61. Haklar, G., Ulukaya-Durakbasa, C., Yuksel, M., Dagli, T., and Yalein, A. S. (1998) Clin. Exp. Pharmacol. Physiol. 25,908-912

We claim:

1. A method for enhancing molecular chaperone activity of α-crystalline (comprising of forms α-crystalline and αB-crystalline) with a biological compatible amino acid molecule of Arginine Hydrochloride, said method comprising the steps of:
(a) isolating and purifying α-crystalline from calf eye lenses by convention methods (as described in reference 24), and
(b) reacting α-crystalline in the presence of phosphate buffer of pH 7.4 with Arg.HCl and insulin or ξ-crystalline in presence or absence of DTT, and
(c) observing the enhancement in chaperone activity of α-crystalline in presence of Arg.HCl by fluorescence spectrophotometer.

2. A method as claimed in claim 1, wherein Arginine hydrochloride (Arg.HCl) binds to the peptide backbone and negatively charged side chains of α-crystalline to enhance chaperone activity.

3. A method as claimed in claim 1, wherein Arg.HCl is in the range of about 50 to 350 mM.

4. A method as claimed in claim 3, wherein Arg.HCl is in the range of about 100 to 300 mM.

5. A method as claimed in claim 1, wherein Arg.HCl enhances the chaperone activity of α-crystalline by about 95%.

6. A method as claimed in claim 5, wherein Arg.HCl enhancs the chaperone activity of α-crystalline by about 90%.

7. A method as claimed in claim 1, wherein Arg.HCl enhance the chaperone activity of α-crystalline by about 90% in presence of various aggregation systems.

8. A method as claimed in claim 7, wherein Arg.HCl enhance the chaperone activity of α-crystalline by about 81% in presence of various aggregation systems.

9. A method as claimed in claim 1 and 7, wherein aggregation systems maybe selected from group comprising of insulin, ξ-crystallin and related compounds.

10. A method as claimed in claim 1, wherein Arg.HCl at a temperature of about 30° C. protects the α-crystalline by about 35%.

11. A method as claimed in claim 1, wherein Arg.HCl at a temperature of about 30° C. protects the α-crystalline by about 28%.

12. A method as claimed in claim 1, wherein Arg.HCl brings about subtle changes in the tertialry structure and significant changes in the quaternary structure of both homo-multimers or hetero-multimers of αA-crystalline and αB-crystalline to enhance the chaperone activity.

13. A method as claimed in claims 1 and 12, wherein presence of Arg.HCl the molecular mass of α-crystalline is reduced ˜360 kDa thereby bringing about subtle changes in the tertialry structure and significant changes in the quaternary structure of both homo-multimers or hetero-multimers of αA-crystalline and αB-crystalline to enhance the chaperone activity.

14. A method as claimed in claim 1, wherein wild type and mutant αA-crystalline are less sensitive to Arg.HCl than αB-crystalline, thereby enhancing the chaperone activity.

15. A method as claimed in claims 1 and 14, wherein protection of mutant αB-crystalline (R120αB-crystallin) is about 80% in presence of Arg.HCl.

16. A method as claimed in claim 15, wherein protection of mutant αB-crystalline (R120αB-crystallin) is about 75% in presence of Arg.HCl.

17. A method as claimed in claim 1, wherein Arg.HCl enhances the α-crystalline chaperone activity by about 45% by exposing the hydrophobic surfaces of α-crystalline.

18. A method as claimed in claim 14, wherein Arg.HCl enhances the α-crystalline chaperone activity by about 38% by exposing the hydrophobic surfaces of α-crystalline.
